(19) United States
(12) Patent Application Publication
(10) Pub. No.: US 2024/0347163 A1
(43) Pub. Date: Oct. 17, 2024

(54) METHODS AND SYSTEMS FOR OBTAINING AND/OR RECONSTRUCTING SENSOR DATA FOR PREDICTING PHYSIOLOGICAL MEASUREMENTS AND/OR BIOMARKERS

(71) Applicant: University of Washington, Seattle, WA (US)

(72) Inventors: Megan Ebers, Seattle, WA (US); Katherine Muterspaugh Steele, Seattle, WA (US); Jose Nathan Kutz, Seattle, WA (US); Jan Williams, Seattle, WA (US)

(21) Appl. No.: 18/632,126

(22) Filed: Apr. 10, 2024

Related U.S. Application Data
(60) Provisional application No. 63/458,850, filed on Apr. 12, 2023.

(51) Int. Cl. G16H 20/30; G16H 50/20

(52) U.S. Cl. CPC G16H 20/30; G16H 50/20

(57) ABSTRACT

This disclosure, in part, discusses systems and/or methods for obtaining a time series of measurements from an at least one mobile sensor carried by a portion of a body during motion of at least one mobile sensor. The systems and/or the methods are used to reconstruct additional estimated sensor data using a machine learning model based on the time series of measurements. The systems and/or methods are used to analyze the additional estimated sensor data together with the time series of measurements to predict a physiological measurement and/or a biomarker.

### CROSS-REFERENCE TO RELATED APPLICATIONS

This application claims the benefit under 35 U.S.C. § 119 (e) of the earlier filing date of U.S. Provisional Application No. 63/458,850, filed Apr. 12, 2023, the entire contents of which are hereby incorporated by reference in their entirety for any purpose.

### STATEMENT OF FEDERALLY SPONSORED RESEARCH AND DEVELOPMENT

This invention was made with government support under Grant Nos. 2112085 and DGE-1762114 awarded by the National Science Foundation. The government has certain rights in the invention.

### BACKGROUND

Certain biomechanical variables have been established as biomarkers (e.g., digital biomarkers) that correlate with meaningful outcomes. The biomarkers may include knee adduction angle(s) for an anterior cruciate ligament (ACL) injury, a step width variability associated with risk(s) due to aging of a person (e.g., a user, a patient, an athlete), falling, segment accelerations associated with athletic performance or injury risk, or other factors that adversely affect the health of the person or enhance human performance. Since many persons have mobility-related health issues, musculoskeletal disorders, or other motion-related hindrances, monitoring motion(s) may be important to observe a person's functionality and lifestyle. For human motion to be observed in natural or uncontrolled environments, sensing devices must be portable, unobtrusive, reliable, and/or accurate. However, for sensing data to be meaningful, measurements must be converted to, and contextualized as, personalized biomechanical outcomes, which can be challenging, prohibitively costly, and/or not feasible in natural environments, as supposed to, for example, in a laboratory environment equipped with state-of-the-art equipment and/or operated by highly trained professionals.

While motion tracking is used in clinical, research, computer graphics, and/or sports settings, the spectra of available technologies may vary widely in practicality, accuracy, and/or cost. Some professionals consider an optical motion capture and force plates to be the “gold standard” to comprehensively capture kinematics (e.g., motion(s)) and kinetics (e.g., force(s)). However, these motion capture cameras and sensors are highly specialized and may require a dedicated laboratory space and/or careful calibration. These systems and methods may only benefit persons who live or work nearby such laboratories, and/or affluent persons. Conversely, more portable devices offer opportunities for out-of-lab motion tracking and across many repetitions, including inertial measurement units (IMUs), electromyography (EMG), depth cameras, red-green-blue (RGB) cameras, pedometers, and/or pressure insoles. Such portable devices can also be used in conjunction with laboratory motion capture.

IMUs generally refer to electronic devices that can be utilized for measuring and/or reporting force, angular rate, and/or magnetic field of a body, a portion of the body, or an object. IMUs can do so by utilizing a combination of accelerometers, gyroscopes, and magnetometers. These data can be used for tracking and analyzing the motion and/or the orientation of the body, the portion of the body, or the object. IMUs may have applications in various fields, such as motion capture, navigation, robotics, biomechanics, or other fields. The resulting information from IMUs aids in understanding the movement patterns and/or dynamics of objects or individuals in controlled or uncontrolled environments.

While portable devices, such as wearable sensors, IMUs, etc., can estimate biomechanical variables like kinematics in any environment, these devices tend to be less accurate than optical motion capture. For example, IMUs are a widely adopted wearable device for portable motion tracking. Yet, questions of reliable placement by non-experts (e.g., users) and error accumulation over long usage to extract meaningful outcomes with IMUs remain a challenge, especially in natural environments. Also, in some instances, ferromagnetic disturbances may erroneously affect magnetometer data collected by the IMUs. Portable devices, such as wearable sensors, may be used for tracking human motions in natural environments. However, data collected by these devices need to be linked to biomechanical outcomes to provide actionable insight for monitoring, diagnosing, and/or treating mobility-related challenges.

Machine learning models can allow researchers and clinicians in sensing and analyzing human motion using wearable sensors, such as wearable sensors with IMUs. Machine learning models can be useful in extracting salient features and modeling complex relationships from the deluge of data collected from laboratory experiments, rehabilitation clinics, and/or wearable sensors.

### SUMMARY

Example methods are disclosed herein. In an embodiments, an example method may include obtaining a time series of measurements from at least one mobile sensor carried by a portion of a body during motion of the at least one mobile sensor. The method may include reconstructing additional estimated sensor data using a machine learning model based on the time series of measurements. The method may include analyzing the additional estimated sensor data together with the time series of measurements to predict a physiological measurement, a biomarker, or combinations thereof.

Additionally, or alternatively, the analyzing of the method may further include selecting a subset of the additional estimated sensor data together with the time series of measurements, where the selected subset of the additional estimated sensor data is related to the physiological measurement, the biomarker, or combinations thereof.

Additionally, or alternatively, the method may further include determining at least one higher-dimensional body movement compared to the time series of measurements from the at least one mobile sensor carried by the portion of the body.

Additionally, or alternatively, the machine learning model may be or include a shallow recurrent decoder neural network.

Additionally, or alternatively, the method may further include training the machine learning model using a plurality of users during a first time period.

Additionally, or alternatively, the obtaining, the reconstructing, and/or the analyzing of the method are associated with a single user during a second time period.

Additionally, or alternatively, an identity of the single user differs from identities of each user of the plurality of users.

Additionally, or alternatively, the method may further include training the machine learning model using a user during a first time period, a first environmental setting, or combinations thereof.

Additionally, or alternatively, the obtaining, the reconstructing, and/or the analyzing of the method are associated with the user during a second time period, a second environmental setting, or combinations thereof.

Additionally, or alternatively, the at least one mobile sensor includes inertial measurement units.

Additionally, or alternatively, the at least one mobile sensor is embedded in a wearable device.

Additionally, or alternatively, the physiological measurement, the biomarker, or combinations thereof comprise a stride length.

Example computing systems are disclosed herein. In an embodiment, an example computing system may include a processor and a non-transitory computer-readable storage medium (a). The computer-readable storage medium may store instructions that, when executed by the processor, cause the computing system to perform one or more operations. The operations may include obtain a time series of measurements from an at least one mobile sensor carried by a portion of a body during motion of the at least one mobile sensor. The operations may include reconstruct additional estimated sensor data using a machine learning model based on the time series of measurements. The operations may include analyze the additional estimated sensor data together with the time series of measurements to predict a physiological measurement, a biomarker, or combinations thereof.

Additionally, or alternatively, the machine learning model may include a time sequence model and a decoder, and the time sequence model may increase an accuracy of the reconstructed additional estimated sensor data.

Additionally, or alternatively, the portion of the body may belong to a user, and the instructions, when executed by the processor, cause the computing system to perform operations that may include map biomechanical measurements during motion of the portion of the body of the user; and train the machine learning model to reconstruct the additional estimated sensor data for the user based on the time series of measurements from the at least one mobile sensor.

Additionally, or alternatively, the instructions, when executed by the processor, cause the computing system to perform operations that may include: map biomechanical measurements of a plurality of users; train the machine learning model to reconstruct the one or more additional estimated sensor data; and/or analyze the additional estimated sensor data together with the time series of measurements to predict the physiological measurement, the biomarker, or combinations thereof of another user. An identity of the other user differs from an identity of each user of the plurality of users, and the portion of the body may belong to the other user.

Additionally, or alternatively, the computing system may further include a display screen to display the physiological measurement, the biomarker, or combinations thereof.

Additionally, or alternatively, the computing system may include a mobile electronic device, where the mobile electronic device may include the at least one mobile sensor.

Additionally, or alternatively, the computing system may further include an interface, where the interface communicatively couples the computing system to the at least one mobile sensor.

Additionally, or alternatively, the at least one mobile sensor may include a thermocouple, a thermistor, a resistance temperature detector, a pressure sensor, a light sensor, a motion sensor, a proximity sensor, a gas sensor, an air quality sensor, a pH sensor, a humidity sensor, a magnetic sensor, a biometric sensor, inertial measurement units, or combinations thereof.

### BRIEF DESCRIPTION OF THE DRAWINGS

FIG. 1 is a schematic illustration of a system arranged in accordance with examples described herein.

FIG. 2 is a flowchart of a method arranged in accordance with examples described herein.

FIG. 3 is a schematic illustration of a machine learning model arranged in a system in accordance with examples described herein.

FIG. 4 is another schematic illustration of a machine learning model arranged in accordance with examples described herein.

FIG. 5 is a schematic illustration of sensors on a user arranged in accordance with examples described herein.

FIG. 6A shows a first graph of measurements of a right ankle dorsiflexion angle, where the measurements may be part of a kinematics dataset, in accordance with examples described herein.

FIG. 6B shows a second, a third, and a fourth graph of reconstructed and measured angles of various body parts of the user, where the reconstructed angles are outputs of the machine learning model, and where the reconstructed and measured angles may be part of the kinematics dataset, in accordance with examples described herein.

FIG. 6C shows a fifth, a sixth, and a seventh graph of reconstructed and measured angles of additional various body parts of the user, where the reconstructed angles are outputs of the machine learning model, and where the reconstructed and measured angles may be part of the kinematics dataset, in accordance with examples described herein.

FIG. 7A shows an eighth graph of measurements of a right wrist acceleration of the user, and a ninth graph of measurements of a left wrist acceleration of the user, where the measurements may be part of an IMU dataset, in accordance with examples described herein.

FIG. 7B shows a tenth, an eleventh, and a twelfth graph of reconstructed accelerations and measured accelerations of various body parts of the user, where the reconstructed accelerations are outputs of the machine learning model, and where the reconstructed and measured accelerations may be part of the IMU dataset, in accordance with examples described herein.

FIG. 7C shows a thirteenth and a fourteenth graph of reconstructed accelerations and measured accelerations of additional various body parts of the user, where the reconstructed accelerations are outputs of the machine learning model, and where the reconstructed and measured accelerations may be part of the IMU dataset, in accordance with examples described herein.

FIG. 8 depicts numbered mathematical equations or expressions which may be referred to herein.

### DETAILED DESCRIPTION

Examples described herein include systems and/or methods for obtaining a time series of measurements from at least one mobile sensor carried by a portion of a body during motion of the mobile sensor. Additionally, or alternatively, the systems and/or methods may obtain the time series of measurements from at least one stationary sensor that is capable of capturing motion of one or more portions of the body.

In some embodiments, the systems and/or methods may reconstruct additional estimated sensor data using, for example, a machine learning model, based on the time series of measurements. The additional estimated sensor data may provide additional insights of the motions, without utilizing additional physical mobile sensors and/or physical stationary sensors. The systems and/or methods may analyze the additional estimated sensor data together with the time series of measurements to predict a physiological measurement, a biomarker, another measurement, or combinations thereof.

In this manner, a first set of sensor data may be obtained from sensors that sense motion of a user. The first set of sensor data may be used to generate a second, generally larger, set of data. This second set of data may be, for example, an expanded set of sensor data. Data from one or more sensors may accordingly be used by a machine learning model described herein to generate another set of data representing additional sensor data. Although the additional sensors were not carried, worn, or otherwise used to sense actual motion of the user, the sensor data may nonetheless be generated by a machine learning model using the input data of a different set of sensor data (generally, a more limited set of sensor data) obtained from sensing the user. The second set of data may be analyzed to determine various biomarkers (e.g., metrics). For example, an analysis technique may utilize a particular set of sensor data to determine a biomarker. However, that set of sensor data may not be available from a user who is utilizing a different set of sensors, such as one sensor or a limited set of sensors. Accordingly, data from the single sensor or limited set of sensors herein may be used to generate reconstructed (e.g., predicted and/or estimated) data from additional sensors. The reconstructed data may be in a format expected from an additional sensor. The reconstructed data may have values expected from an additional sensor. However, the additional sensor may not have been used to actually sense the user. Having generated reconstructed data from a sensor of interest, the technique for determining a biomarker using data from the sensor of interest may now be used on the reconstructed data.

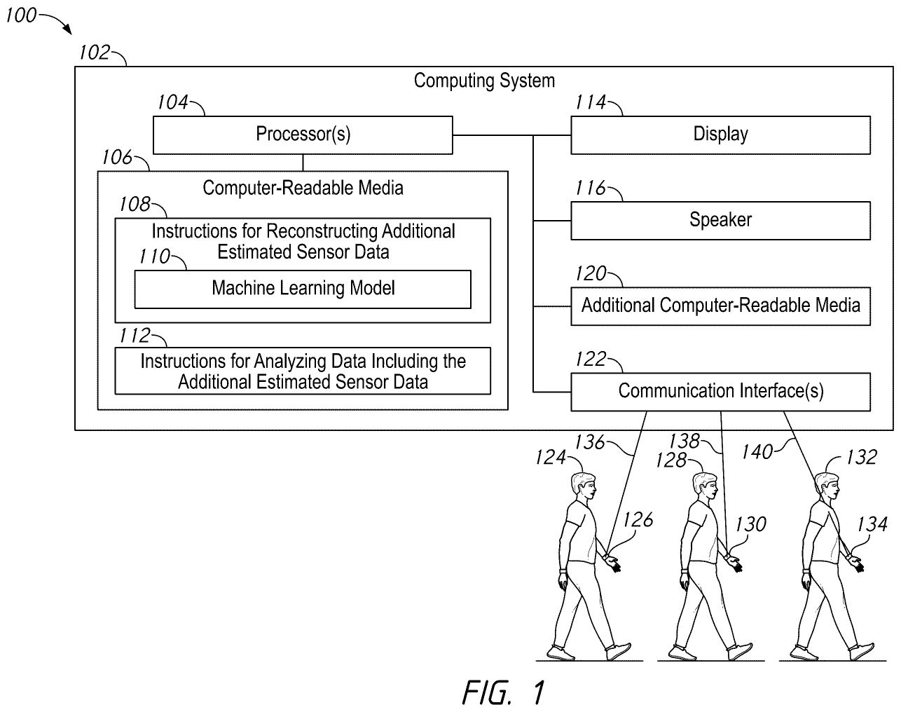

FIG. 1 is a schematic illustration of a system 100 arranged in accordance with examples described herein. FIG. 1 depicts a computing system 102 communicatively coupled to mobile sensor(s) 126, mobile sensor(s) 130, and mobile sensor(s) 134 that are being utilized by a user 124, a user 128, and a user 132, respectively, in accordance with examples described herein. Additionally, or alternatively, the computing system 102 may be communicatively coupled to a stationary sensor(s). The system may be located in a natural environment, an every-day environment, or a laboratory environment.

Although not illustrated as such in FIG. 1, in some embodiments, the environment may include any count (e.g., number) of users, including 1, 2, 3, 4, 5, 6, 7, 8, 9, or more users, where each user may utilize one or a plurality of mobile sensors. In some embodiments, the users may not be humans, such as pets, or other creatures in the kingdom Animalia. Although not illustrated as such, depending on the application, the sensors may be embedded in or on inanimate or physical objects, such as mountains, hills, fields, rivers, seas, oceans, icebergs, etc.

In some embodiments, the computing system 102 may include or utilize a processor(s) 104, a computer-readable media 106, instructions for reconstructing additional estimated sensor data 108, machine learning model 110, instructions for analyzing data including the additional estimated sensor data 112, a display 114, a speaker 116, an additional computer-readable media 120, and communication interface(s) 122. In some embodiments, the computing system 102 may include fewer, additional, and/or different components than what is shown in FIG. 1.

In some embodiments, the computing system 102 may communicate with the mobile sensor(s) 126, the mobile sensor(s) 130, and/or the mobile sensor(s) 134 using the communication coupling 136, the communication coupling 138, and/or communication coupling 140, respectively.

In some embodiments, the computing system 102, any of the mobile sensors of FIG. 1, and/or other stationary sensors (not illustrated in FIG. 1) may be packaged as an integrated unit. In such as case, any of the sensors may be included in a single computing system (not illustrated as such in FIG. 1). For example, the computing system 102 may include the sensor 126 in some examples.

In some embodiments, communication(s) and/or communication coupling(s) in the environment 100 of FIG. 1 may be (or performed using) various protocols and/or standards. Examples of such protocols and standards include: a 3rd Generation Partnership Project (3GPP) Long-Term Evolution (LTE) standard, such as a 4th Generation (4G) or a 5th Generation (5G) cellular standard; an Institute of Electrical and Electronics (IEEE) 802.11 standard, such as IEEE 802.11g, ac, ax, ad, aj, or ay (e.g., Wi-Fi 6® or WiGig®); an IEEE 802.16 standard (e.g., WiMAX®); a Bluetooth Classic® standard; a Bluetooth Low Energy® or BLE® standard; an IEEE 802.15.4 standard (e.g., Thread® or ZigBee®); other protocols and/or standards that may be established and/or maintained by various governmental, industry, and/or academia consortiums, organizations, and/or agencies; and so forth.

Therefore, in some embodiments, the computing system 102 may communicate with the mobile sensor(s) 126, 130, 134, stationary sensors (not illustrated in FIG. 1) and/or other devices that are capable of performing measurements (e.g., a time series of measurements). These communications may be performed utilizing a cellular network, the Internet, a wide area network (WAN), a local area network (LAN), a wireless LAN (WLAN), a wireless personal-area-network (WPAN), a mesh network, a wireless wide area network (WWAN), a peer-to-peer (P2P) network, and/or a Global Navigation Satellite System (GNSS) (e.g., Global Positioning System (GPS), Galileo, Quasi-Zenith Satellite System (QZSS), BeiDou, GLObal NAvigation Satellite System (GLONASS), Indian Regional Navigation Satellite System (IRNSS), and so forth). In addition to, or alternatively of, the communications illustrated in FIG. 1, the environment 100 may facilitate other unidirectional, bidirectional, wired, wireless, direct, and/or indirect communications utilizing one or more communication protocols and/or standards. Therefore, FIG. 1 does not necessarily illustrate all communication signals.

In some embodiments, the computing system 102 may be implemented using combinations of hardware, software, firmware, and/or data that work together to perform computing tasks. The computing system 102 may be or include a personal computing system, a server computing systems, an embedded computing system, a mainframe computing system, a cloud computing system, and so forth. The computing system 102 may be mobile or stationary. For example, the computing system 102 may be implemented using one or more servers, desktops, laptops, tablets, smartphones, smart speakers, appliances, vehicles, or combinations thereof.

In some embodiments, the processor(s) 104 may be implemented using an electronic device that may be capable of processing, receiving, and/or transmitting instructions that may be included in, permanently or temporarily saved on, and/or accessed by the computer-readable media 106, the additional computer-readable media 120, or another computer-readable media that is not illustrated in FIG. 1. In aspects, the processor(s) 104 may be implemented using one or more processors (e.g., a central processing unit (CPU), a graphic processing unit (GPU)), and/or other circuitry, where the other circuitry may include at least one or more of an application specific integrated circuit (ASIC), a field programmable gate array (FPGA), a microprocessor, a microcomputer, and/or the like. Furthermore, the processor(s) 104 may be configured to execute the instructions for reconstructing additional estimated sensor data 108, the machine learning model 110, the instructions for analyzing data including the additional estimated sensor data 112, or other instructions, in parallel, locally, and/or across a network, for example, by using cloud and/or server computing resources.

As is illustrated in FIG. 1, the processor(s) 104 may execute instructions to operate, associated with, and/or part of, the computer-readable media 106, the instructions for reconstructing additional estimated sensor data 108, the machine learning model 110, the instructions for analyzing data including the additional estimated sensor data 112, the display 114, the speaker 116, communication interface(s) 122, and/or other components and/or entities of, or coupled with, the computing system 102 that may not be explicitly illustrated in FIG. 1.

In some embodiments, the computer-readable media 106 and/or the additional computer-readable media 120 may be and/or include any data storage media, such as volatile memory and/or non-volatile memory. Examples of volatile memory may include a random-access memory (RAM), such as a static RAM (SRAM), a dynamic RAM (DRAM), or a combination thereof. Examples of non-volatile memory may include a read-only memory (ROM), a flash memory (e.g., NAND flash memory, NOR flash memory), a magnetic storage medium, an optical medium, a ferroelectric RAM (FeRAM), a resistive RAM (RRAM), and so forth.

In some embodiments, the instructions for reconstructing additional estimated sensor data 108 and/or the instructions for analyzing data including the additional estimated sensor data 112 may be included in, permanently or temporarily saved on, and/or accessed by the computer-readable media 106 of the computing system 102. The instructions for reconstructing additional estimated sensor data 108 and/or instructions for analyzing data including the additional estimated sensor data 112 may include code, pseudo-code, algorithms, the machine learning model 110, other models, software, and/or so forth and may be executable by the processor(s) 104.

Although, the machine learning model 110 is illustrated as being stored in the computer-readable media 106 of the computing system 102, in some embodiments, the machine learning model 110 may be located outside the computer-readable media 106 and/or the computing system 102. For example, the machine learning model 110 may be stored and/or trained on a server or a cloud that is not part of the computing system 102. The computing system 102 and/or the computer-readable media 106 may access the machine learning model 110 using the communication interface(s) 122.

In some embodiments, the display 114 may display visual information, such as an image(s), a video(s), a graphical user interface (GUI), notifications, instructions, text, and/or so forth. The display 114 may aid the user (e.g., the user 124, the user 128, the user 132, a patient, a trainer, a patient, a medical professional) in interacting with the computing system 102, any of the mobile sensors illustrated in FIG. 1, a stationary sensor (not illustrated in FIG. 1), and/or another device (not illustrated in FIG. 1). In some embodiments, the display 114 may display images and/or instructions requesting user input (e.g., via a GUI) during the training of the machine learning model 110; and/or the operation of the computing system 102, any of the mobile sensor(s), stationary sensor(s), and/or any other device. In some embodiments, the display 114 may display one or more biomarkers and/or output of analysis described herein.

The machine learning model 110 may in some examples be implemented using two components. A first component in the machine learning model 110 may be a sequential model for encoding time sequences. Accordingly, all or a portion of the machine learning model 110 may encode time sequences of sensor data as described herein. Examples of the sequential model include, but are not limited to a long short-term memory (LSTM) network, a recurrent neural network (RNN), and/or a network using one or more gated recurrent units (GRUs). A second component in the machine learning model 110 may be a decoder which may map a latent space of time encoder to spatial decoding. For example, the decoder component may receive an output of the first component and may map the output to a final output of the machine learning model 110. In this manner, an encoded time sequence may be used to map to a final spatial output.

In some embodiments, the display 114 may utilize a variety of display technologies, such as a liquid-crystal display (LCD) technology, a light-emitting diode (LED) backlit LCD technology, a thin-film transistor (TFT) LCD technology, an LED display technology, an organic LED (OLED) display technology, an active-matrix OLED (AMOLED) display technology, a super AMOLED display technology, and so forth. In some embodiments, the computing system 102 may be an AR/VR headset. In such a case, the display 114 may also include a transparent or semi-transparent element, such as a lens or waveguide, that allows the user to simultaneously see a real environment and information or objects projected or displayed on the transparent or semi-transparent element, such as virtual objects in a virtual environment.

In some embodiments, the speaker 116 may read aloud words, phrases, and/or instructions provided by the computing system 102, and the speaker 116 may aid the user in interacting with the computing system 102, any of the mobile sensors illustrated in FIG. 1, a stationary sensor (not illustrated in FIG. 1), and/or other devices (not illustrated in FIG. 1). In some embodiments, the speaker 116 may read aloud words, phrases, and/or instructions requesting user input during the training of the machine learning model 110; and/or the operation of the computing system 102, any of the mobile sensor(s), stationary sensor(s), and/or any other device.

In some embodiments, the mobile sensor(s) 126, the mobile sensor(s) 130, and/or the mobile sensor(s) 134 may be or include a wide variety of sensors designed to measure, detect, and/or record different physical phenomena. For example, any measurement device that can record a time sequence of measurements may be used. The mobile sensors may be or include one or more temperature sensors, thermocouples, thermistors, resistance temperature detectors (RTDs), pressure sensors, light sensors, motion sensors, proximity sensors, gas sensors (e.g., carbon monoxide sensors, air quality sensors, etc.), pH sensors, humidity sensors, magnetic sensors, biometric sensors, etc. In some embodiments, the mobile sensors may be or include IMUs having one or more accelerometers, gyroscopes, and/or magnetometers.

In some embodiments, communication interface(s) 122 may allow the computing system 102 to receive an input(s) from the user 124, the user 128, the user 132, another user, and/or a plurality of other users, where each user may carry, wear, and/or utilize at least one mobile sensor (e.g., as is illustrated in FIG. 1) and/or at least one stationary sensor (e.g., not illustrated as such in FIG. 1). The inputs may be specified by the user and/or may be automatically provided by the mobile sensor(s) 126, the mobile sensor(s) 130, the mobile sensor(s) 134, and/or a stationary sensor(s).

In some embodiments, the mobile sensors 126, 130, and/or 134 can measure a time series of measurements and send these measurements to the computing system 102 using the communication couplings 136, 138, and/or 140. The time series may include a continuous set of measurements and/or a set of discrete measurements. For example, a number of sensor readings may be provided over a period of time. For example, a sensor value may be communicated periodically. The sensor values may be continuous in some examples and/or discrete in some examples. For example, sensor values may be provided continuously over a period of time, such as over 30 seconds, 1 minute, 5 minutes, 10 minutes, 30 minutes, 1 hour, 2 hours, 5 hours, 10 hours, 12 hours, 24 hours, one day, one week, and/or one month. Other intervals may be used in other examples. In some examples, discrete sensor values may be used. A sensor value may be communicated from the sensor periodically, such as every millisecond, every second, every 30 seconds, every minute, every 5 minutes, every 10 minutes, every 30 minutes, every hour, every 2 hours, and/or every 5 hours. Other intervals may be used in some examples. In some examples, the sensor values may not be reported at regular intervals. For example, sensor values may be communicated responsive to particular values or ranges being sensed.

Generally, mobile sensors described herein may be carried by a portion of a user's body. The mobile sensor may be carried by, for example, being worn, supported by, attached to, and/or coupled to a portion of the user's body. The mobile sensor may be carried by, for example, being supported by, attached to, and/or couple to another article which is carried by the user. The mobile sensor may be, for example, disposed in a purse, bag, briefcase, box, or other receptacle. A variety of body portions may be used to carry mobile sensors described herein including one or more heads, foreheads, arms, legs, ankles wrists, toes, fingers, chests, cars, and/or hips.

In some embodiments, the mobile sensor(s) 126, the mobile sensor(s) 130, and/or the mobile sensor(s) 134 may be embedded in or on an electronic device, which may be a wearable device such as a smartwatch, a fitness tracker, a health and/or wellness wearable device, a sports performance wearable device, a medical wearable device, an outdoor adventure wearable device, smart clothing and/or e-textiles, and/or so forth. Examples of wearable devices include, but are not limited to, one or more watches, wristbands, headbands, socks, shirts, pants, belts, rings, necklaces, bracelets, eyeglasses, and/or hats. Other electronic devices which may be used include smartphones, GPS units, AR/VR headsets.

In some embodiments, the computing system 102, the mobile sensor(s) 126, 130, 134, stationary sensors (not illustrated in FIG. 1), and/or any other system(s) and/or method(s) described herein may meet or exceed industry best-practices. For example, in the USA, if these systems and/or methods are used in the medical field, they may meet and/or exceed the medical standards established by the Health Insurance Portability and Accountability Act of 1996 (HIPAA). As another example, internationally, if these systems and/or methods are used in sports training and/or sports medicine, they may meet or exceed the standards set by, for example, the Fédération Internationale de Football Association (FIFA) and/or any other international sports association. Therefore, depending on the application, these systems and/or methods may meet and/or exceed any standards established and/or adopted in local, state, country, and/or international jurisdictions.

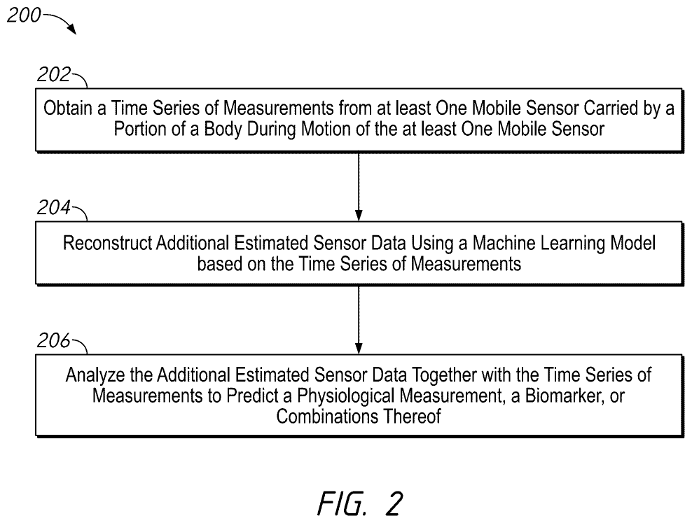

FIG. 2 shows a block diagram of a method 200 for predicting a physiological measurement and/or a biomarker, in accordance with examples described herein. In some embodiments, the method 200 of FIG. 2 may be implemented using at least the computing system 102, the mobile sensor(s) 126, 130, 134, and/or any other device illustrated and/or described with reference to FIG. 1. The blocks in FIG. 2 are exemplary. Additional, fewer, and/or different blocks may be present in other examples. In some examples, the blocks may occur in different orders and/or at least in part simultaneously.

Biomarkers herein generally refer to indicators of biological processes, disease states, responses to therapeutic interventions, and/or so forth. Biomarkers can play a crucial role in various fields, including medicine, pharmacology, environmental science, or other fields. Biomarkers can be categorized into different types, in part, based on their applications, sources, and/or characteristics. Example types of biomarkers, include diagnostic biomarkers, prognostic biomarkers, predictive biomarkers, pharmacodynamic biomarkers, surrogate biomarkers, environmental biomarkers, genomic biomarkers, proteomic biomarkers, metabolomic biomarkers, and/or so forth.

Physiological measurements herein may refer to the process of quantitatively and/or qualitatively assessing various physiological parameters and/or functions in humans or other living organisms. These measurements may be used for evaluating the health, performance, and/or responses of biological systems. Thus, the physiological measurements may provide valuable insights into the body's functioning at a molecular, cellular, organ, and/or systemic level. Physiological measurements may include a wide range of quantitative assessments, including: vital signs, such as body temperature, heart rate, respiratory rate, blood pressure, and/or so forth; electrocardiogramhy (ECG or EKG) for recording and/or analyzing the electrical activity of the heart; spirometry, such as lung volume, capacity, and/or airflow rates; blood tests, such as glucose, cholesterol, hormones, enzymes, cellular elements, and/or so forth; neurological measurements, such as electroencephalography (EEG); physical performance tests, such as endurance, strength, flexibility, balance, coordination, and/or so forth; imaging and/or diagnostic tests with the aid of medical imaging techniques and/or equipment; and/or other physiological measurements.

In some embodiments, at block 202, the method 200 may include obtaining a time series of measurements from at least one mobile sensor carried by a portion of a body during motion of the at least one mobile sensor. The mobile sensor may be or include the mobile sensor(s) 126, 130, 134 of FIG. 1, and/or other mobile sensors from the same users and/or other users.

Alternatively, or additionally, the time series of measurements may be obtained from at least one stationary sensor. For example, the method 200 may be implemented in a laboratory environment equipped with state-of-the-art equipment and/or operated by highly trained professionals. In some embodiments, the laboratory environment may be useful in training the machine learning models 110 of FIG. 1. However, the laboratory environment is not required to train the machine learning model 110 of FIG. 1. The machine learning model 110 of FIG. 1 may be trained in some examples from sensors carried and/or used by users in natural, every-day, and/or laboratory environments.

Accordingly, in block 202, one or more time series of measurements from sensors 126, 130 and/or 134 of FIG. 1 may be communicated to the computing system 102. In some examples, some or all of the obtained time series measurements may be stored at the computing system 102 or in electronic storage accessible to the computing system 102, such as in computer-readable media 106 and/or additional computer-readable media 120.

In some embodiments, at block 204, the method 200 may include reconstructing additional estimated sensor data using a machine learning model based on the time series of measurements obtained from the mobile sensor(s). The machine learning model of block 204 of FIG. 2 may be or include the machine learning model 110 of FIG. 1. At block 204, the method 200 may include expanding the quantity of sensor data, even though these additional estimated sensor data may not be explicitly measured by physical sensors. In some embodiments, block 204 of the method 200 of FIG. 2 is described in the context of the instructions for reconstructing additional estimated sensor data 108 of FIG. 1. The additional estimated sensor data may be sensor data of other sensors, different from those used in block 202, in some examples. In some examples, the additional estimate sensor data may be or may include estimated sensor data from one or more of the sensors used in block 202, but may be estimated at one or more times during which the sensors used in block 202 did not sense the user and/or sensed a different user.

Accordingly, at block 204, the computing system 102 may reconstruct the additional estimated sensor data in accordance with the instructions for reconstructing additional estimated sensor data 108 of FIG. 1, which may be executed by processor(s) 104. For example, the computing system 102 may provide one or more time series measurements from sensors to machine learning model 110. The machine learning model 110 may output the additional estimated sensor data.

In some embodiments, at block 206, the method 200 may include analyzing the additional estimated sensor data together with the time series of measurements to predict a physiological measurement, a biomarker, or combinations thereof. Therefore, the method 200 uses measured sensor data and estimated sensor data to determine (e.g., predict) the physiological measurements and/or the biomarkers. For example, the computing system 102 may analyze the additional estimated sensor data together with the time series of measurements in accordance with the instructions for analyzing data including the additional estimated sensor data 112 of FIG. 1, which may be executed by the processor(s) 104.

Examples of physiological measurements and/or biomarkers which may be determined (e.g., predicted) in accordance with examples described herein include knee joint angle, oxygen level, and/or step width. Other examples include running parameters, walking parameters, and/or risk of fall.

In some embodiments, the method 200 may also include selecting a subset of the additional estimated sensor data together with the time series of measurements, where the selected subset of the additional estimated sensor data is related to the physiological measurement, the biomarker, or combinations thereof. For example, some of the reconstructed additional estimated sensor data at block 204 may be irrelevant, or have less relevance, in predicting a specific physiological measurement and/or biomarker. Therefore, in some embodiments, at block 206, the method 200 may initially determine which of the additional estimated sensor data can be useful for predicting a specific physiological measurement and/or biomarker. After the method 200 determines the subset of the additional estimated sensor data, the method 200 can be used to predict the physiological measurement and/or the biomarker by using the subset of the additional estimated sensor data together with the time series of measurements. Additionally, or alternatively, the method 200 may determine the subset of the additional estimated sensor data and predict the physiological measurements and/or biomarkers at the same time, instead of, for example, sequentially. Block 206 of the method 200 of FIG. 2 may be described in the context of the instructions for analyzing data including the additional estimated sensor data 112 of FIG. 1.

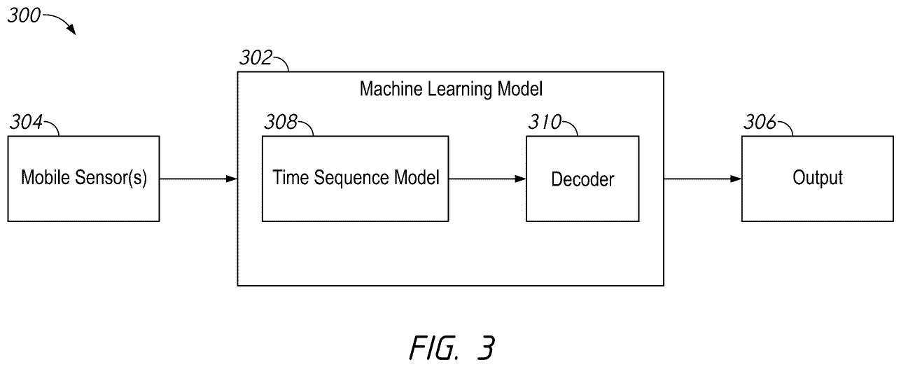

FIG. 3 shows a block diagram 300 of a machine learning model 302, where the input to the machine learning model 302 is data from a mobile sensor(s) 304, and where the machine learning model 302 has an output 306, in accordance with examples described herein. The machine learning model 302 of FIG. 3 may be used to implement and/or implemented by the machine learning model 110 of FIG. 1. Depending on the application, the output 306 may be a physiological measurement, a biomarker, and/or another output.

In some embodiments, the machine learning model 302 may include a time sequence model 308, a decoder 310, or combinations thereof. The machine learning model 302 may include one or more executable instructions and/or data representing weights or other model parameters. Generally, a trained model may be represented by one or more weights for a particular set of executable instructions.

In some embodiments, the time sequence model 308 may analyze and make predictions based on sequential or time-dependent data. The time sequence model 308 may be well-suited for tasks, where the order, timing, and/or interdependencies of the data received from the mobile sensor(s) 304 and/or stationary sensor(s) (not illustrated in FIG. 3). The time sequence model 308 may accordingly increase the accuracy of the analysis of the data and the prediction process. In some embodiments, the prediction process may include predicting a physiological measurement and/or a biomarker. The time sequence model 308 may be implemented using a machine learning model, e.g., one or more executable instructions and data indicative of one or more weights and/or other parameters used by the model.

The machine learning model 302 and/or the time sequence model may be trained. Training generally involves providing the models with training data and utilizing feedback to determine weights or other parameters for use by the model during inference. In examples described herein, machine learning models may be trained on training data which includes a number of sensors worn by users, including in some examples one or more sensors to be worn by users during operation of systems described herein (e.g., an IMU). Accordingly, in some examples, the machine learning model 302 of FIG. 3 may be trained by training data representing ten sensors being worn by users. The ten sensors may be complicated, sensitive, cumbersome and/or expensive sensors in some examples. Machine learning models described herein may be trained to output reconstructed data from a larger number of sensors (e.g., ten sensors) based on input sensed data from a smaller number of sensors (e.g., one sensor). Accordingly, machine learning models described herein, such as time sequence model 308 may receive a time series of measurements from a small set of sensors (e.g., one sensor) during operation, and may be trained to output reconstructed data from a larger number of sensors (e.g., ten sensors). In this manner, a set of sensor may be obtained which represents data that would have been generated had the user been wearing all ten sensors.

Although some of the focus of this description is geared towards predicting a physiological measurement and/or biomarker, it should be understood that the machine learning model 302 and/or the time sequence model 308 may be used in other applications. The other applications may include a time series forecasting or predicting, such as stock prices; weather patterns; ocean “health;” natural language processing (NLP); analyzing and generating text; speech recognition; gesture recognition; signal processing; and/or other applications.

In some embodiments, the time sequence model 308 may be or include a recurrent neural network (RNN); a long short-term memory network (LSTM); a gated recurrent unit (GRU); a specialized RNN architecture that may address the challenge of capturing long-range dependencies in sequential data; a convolutional neural networks (CNN); a modified CNN architecture that may be adapted to process one-dimensional sequential data; an autoregressive integrated moving average (ARIMA); an exponential smoothing state space model (ETS); a WaveNet; an XGBoost; a LightGBM; a CatBoost; and/or any other model suited to analyze and make predictions based on sequential or time-dependent data received from the mobile sensor(s) 304.

The decoder 310 of the machine learning model 302 may depend on the particular type of the time sequence model 308 of the machine learning model 302. In some embodiments, the decoder 310 of the machine learning model 302 may generate the output 306 based on the data of the mobile sensor(s) 304, where the data is encoded by the time sequence model 308 of the machine learning model 302. The decoder 310 may be implemented using one or more executable instructions stored in computer-readable media and/or one or more stored parameters—e.g. stored in computer readable media of FIG. 1.

In some embodiments, if the time sequence model 308 is a sequence-to-sequence (Seq2Seq) model, the time sequence model 308 may process the input sequence from the mobile sensor(s) 304 (or a stationary sensor), and the time sequence model 308 may produce a context or hidden representation that captures the input sequence's information and/or data. In such a case, the decoder 310 may then utilize this context to generate an output sequence of the output 306, which may have a different length or structure than the input sequence.

In some embodiments, if the time sequence model 308 is or includes an RNN, the decoder 310 may use a different RNN structure from the time sequence model 308. For example, if the time sequence model 308 is based on LSTMs or GRUs, the decoder 310 (which operates based on the output of the time sequence model 308) may optionally use the target output of the output 306 in a teacher-forcing manner in training.

In some embodiments, the decoder 310 may include an attention mechanism to allow the machine learning model 302 to focus on different parts of the input sequence from the mobile sensor(s) 304, as the machine learning model 302 generates the output 306. By so doing, the machine learning model 302 may selectively attend to relevant parts of the input sequence from the mobile sensor(s) 304, which can be useful in tasks involving long input sequences or complex dependencies.

In some embodiments, the decoder 310 may generate the output 306 step by step, for example, autoregressively, where each output at a time step t can be used as an input to generate another output at a time step t+1. This process may continue until an end-of-sequence token is generated or a predefined maximum length is reached.

In some embodiments, for example, during training of the machine learning model 302, the decoder 310 may be trained using a teacher-forcing technique, where the true target output of the output 306 may be provided as an input to the decoder 310 at each processing step.

Generally, any of a variety of training mechanisms may be used, including supervised and unsupervised learning. In some examples, models described herein may be trained using training data obtained from the user who will subsequently provide time sequence measurements using one or more sensors described herein. In some examples, models described herein may be trained using training data obtained from other users different from those who will be operating systems described herein during normal operation.

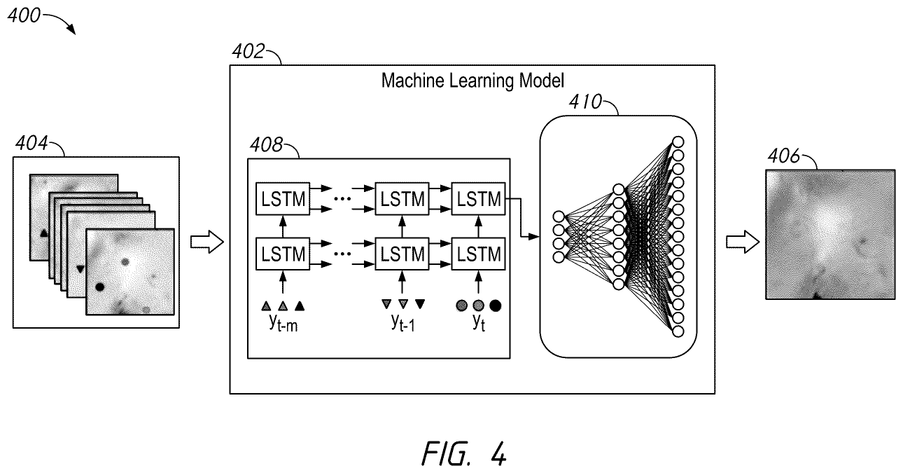

FIG. 4 shows a block diagram 400 of a machine learning model 402 having an input 404 and an output 406, in accordance with examples described herein.

In some embodiments, the machine learning model 402 of FIG. 4 may be used to implement and/or may be implemented by the machine learning model 110 of FIG. 1, the machine learning model used to perform block 204 of FIG. 2, and/or the machine learning model 302 of FIG. 3. In some embodiments, the input 404 of FIG. 4 may be used to implement and/or be implemented by data received from the mobile sensor(s) 126 of FIG. 1, the mobile sensor(s) 130 of FIG. 1, the mobile sensor(s) 134 of FIG. 1, the time series of measurements of block 202 of FIG. 2, data from the mobile sensor(s) 304 of FIG. 3, and/or data from stationary sensors.

In some embodiments, the input 404 may be or include mobile sensor data and/or stationary sensor data. In some embodiments, the output 406 may be or include physiological measurements, biomarkers, and/or other outputs depending on the application.

In some embodiments, the machine learning model 402 may include a time sequence model 408 and a decoder 410. The machine learning model 402 may be or include a shallow recurrent decoder neural network “SHRED” architecture. Generally, a SHRED architecture may refer to a specific type of neural network architecture that may be used in sequence-to-sequence learning tasks. The term “shallow” of the SHRED architecture may refer to the network having a relatively small number of layers (e.g., low count, one, two, three layers, etc.), compared to deeper architectures that may have more layers than the SHRED architecture. The term “recurrent” of the SHRED architecture may refer to the network utilizing RNN layers or variants of the RNN layers.

For example, as is illustrated in FIG. 4, the time sequence model 408 may be an LSTM architecture or an LSTM-based architecture. In some embodiments, the LSTM architecture may be a type of an RNN architecture, and the LSTM architecture may better overcome some of the limitations of traditional (or other) RNN architectures in handling long-term (or relatively long-term) dependencies in sequential data. For example, the LSTM architecture may address a vanishing gradient problem, which can occur when training RNNs on long sequences.

Although not illustrated in exhaustive detail in FIG. 4, some components of the LSTM architecture may include one or more cell states, one or more hidden states, one or more input gates, one or more forget states, one or more output gates, and/or so forth. In some embodiments, the operations performed by these gates may include the use of activation functions, such as the sigmoid and/or tan h functions. These functions can help control the flow of information and/or sensor data. The LSTM architecture can capture and/or record dependencies of the sensor data over longer sequences compared to traditional (or other) RNN architectures. Therefore, the LSTM architecture may be particularly effective for tasks involving time-series data, such as time-series sensor data, the input 404, etc.

It is to be understood that the LSTM architecture is not a limiting example, and the time sequence model 408 of the machine learning model 402 may use other models to process sequential sensor data, such as GRUs, etc. The LSTM architecture and/or the time sequence model 408 may maintain an internal state and may consider each input in the context of previous inputs. The decoder (e.g., the decoder 410) of the SHRED architecture (e.g., the machine learning model 402) may include an encoder-decoder architecture that may be used in sequence-to-sequence tasks. In this architecture, the encoder (e.g., time sequence model 408) may process the input sequence of the input 404, while the decoder (e.g., the decoder 410) may generate the output sequence of the output 406 based on the encoded representation. Generally, the machine learning model 402 and/or the SHRED architecture may leverage recurrent connections (and/or interdependencies) to capture dependencies across input sequences of the input 404, and the machine learning model 402 and/or the SHRED architecture may generate meaningful output sequences of the output 406, with a focus on a more manageable number of network layers compared to deeper architectures.

In some embodiments, the SHRED architecture may use fewer computational and/or storing resources compared to the deeper architectures, without sacrificing or lowering the accuracy of the output 406. Nevertheless, in some embodiments, the machine learning model 402 can be implemented using deeper architectures compared to the SHRED architecture.

In some embodiments, the SHRED architecture and/or the machine learning model 402 may include a neural network mapping from a time history of measurements (e.g., measurements from the mobile sensor(s) 126, 130, 134 of FIG. 1, and/or other mobile or stationary sensors) to, for example, a higher-dimensional and/or spatiotemporal state. For example, the SHRED architecture and/or the machine learning model 402 may determine at least one higher-dimensional body movement compared to the time series of measurements from at least one mobile sensor carried by a portion of a body of a user.

A higher-dimensional body movement generally refers to a movement that may be represented in a larger-dimensional space than the dimensional space in which the mobile sensor carried by the user is operating. For example, in examples described herein, a user may carry only an IMU sensor, such as commonly found in smartwatches or other wearable devices or smartphones. The IMU sensor may generally be able to provide acceleration and/or gyroscope data for a body segment, but ordinarily may not be able to provide insight into acceleration of other body segments, or other metrics like joint angles, stride length, heart rate, or other higher-dimensional movements. For example, the knee angle may indicate the health, flexibility, and/strength of the person's knee. As another example, the stride length may be used to calculate step count or distance travelled more accurately and/or provide information regarding healing or effectiveness of various orthopedic interventions. However, examples described herein utilize machine learning models to reconstruct additional sensor data from which these higher-dimensional body movements may be detected and/or identified.

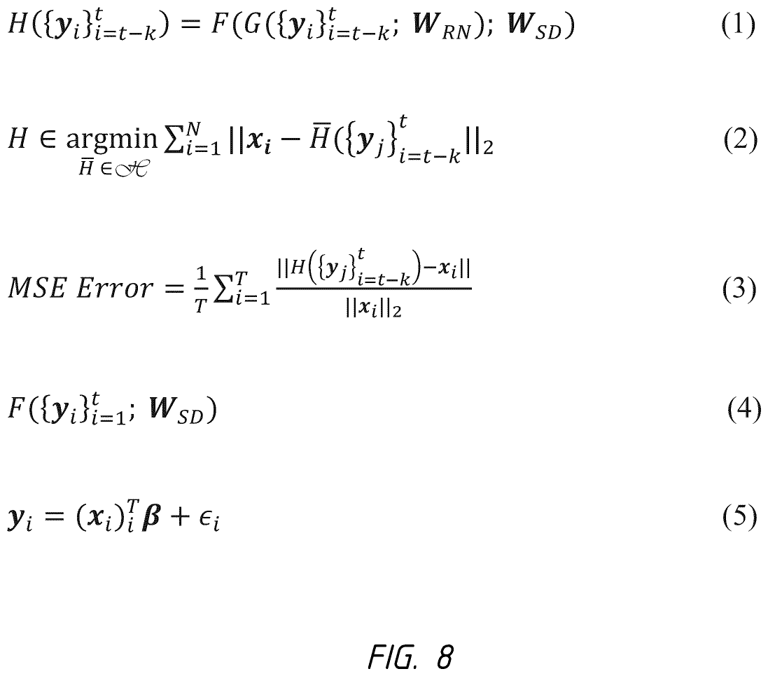

In some embodiments, the SHRED architecture and/or machine learning model 402 may be expressed using equation one “(1)” of FIG. 8. In (1) of FIG. 8, yₜ may denote measurements (e.g., mobile sensors and/or stationary sensors) of a higher-dimensional and/or spatiotemporal state xₜ; F may define a fully-connected and/or feed-forward neural network that may be parameterized by weights W_{SD}; and G may define an LSTM model and/or network that may be parameterized by weights W_{RN}.

In some embodiments, the SHRED architecture's minimized reconstruction loss H may be expressed using the mathematical expression two “(2)” of FIG. 8. In (2) of FIG. 8, {xᵢ}^{N} may denote a set of training states; and {yᵢ}^{N} may denote corresponding measurements of the training states. In some embodiments, the SHRED architecture and/or the machine learning model 402 of FIG. 4 may be trained to minimize the reconstruction loss by using an adaptive movement estimation optimizer “ADAM optimizer.” Generally, the ADAM optimizer can be efficient in optimizing complex models and handling large datasets. The ADAM optimizer may combine features from, for example, the AdaGrad and/or the RMSProp algorithms. The ADAM optimizer can provide adaptive learning rate(s) and momentum (a), resulting in robust performance across a wide range of neural network architectures, such as the SHRED architecture, the LSTM architecture, the time sequence model 408, and/or the machine learning model 402.

In some embodiments, the computing system 102 of FIG. 1 can calculate and/or determine a reconstruction error of the SHRED architecture and/or the machine learning model 402 by calculating an averaged mean square error “MSE error” over each state in a partitioned test set by using equation three “(3)” of FIG. 8. In some embodiments, since the SHRED architecture and/or the machine learning model 402 relies on time series of measurements for state estimation and/or reconstruction (e.g., higher-dimensional and/or spatiotemporal state(s)), each, some, or all of the dataset(s) may be truncated such that the final N-k temporal snapshots are reconstructed, where N may denote the initial number of examples, and k me denote the length of the utilized time history.

Although not illustrated as such in FIG. 4, in some embodiments, the machine learning model 402 may be or utilize other architectures, such as a shallow decoder neural network “SDN” architecture. For example, the SDN architecture, which may be a fully-connect feed-forward neural network, can sensor measurements to a higher-dimensional and/or map spatiotemporal state. However, a difference between the SHRED architecture illustrated in FIG. 4 and the SDN architecture is the fact that the SDN architecture may not utilize the time series of measurements. In some aspects, the SDN architecture may not include an LSTM architecture, a GRU architecture, or another similar architecture that is sometimes used to process sequential data.

In some embodiments, the SDN architecture may be expressed using the mathematical expression four “(4)” of FIG. 8. In (4) of FIG. 8, yₜ may denote measurements of the higher-dimensional and/or spatiotemporal state (e.g., motion, position, angle, etc.) xₜ; and F may denote a fully-connect feed-forward neural network parameterized by weights W_{SD}.

Similar to the SHRED architecture, in some embodiments, the SDN architecture can be trained to minimize reconstruction loss using the ADAM optimizer and/or (3) of FIG. 8.

Although not illustrated as such in FIG. 4, in some embodiments, the machine learning model 402 may be or utilize linear regression and/or another regression-based model. For example, regression-based models, may include a least squares linear regression; least absolute shrinkage and selection operator (LASSO) regression; clastic net regression; linear support vector (SVR) regression; polynomial regression; decision tree regression; random forest regression; gradient boosted regression trees “GBM;” Huber regression, etc.

For example, a least squares linear regression model may be used because it can model the relationship between a dependent variable and one or more independent variables. This regression model can find the best-fitting linear equation that can give insights into association(s) between the predictors and the target variable. The term “least squares” may refer to the approach of minimizing the sum of the squared differences between the observed values and the values estimated by the linear equation. The least squares linear regression model is widely used in various fields, such as economics, medicine, sports, engineering, social sciences, business, etc. to analyze and predict relationships between variables.

In some embodiments, the least squares linear regression model may fit a linear model with coefficients to minimized values of the residual sum of squares between the observed higher-dimensional and/or spatiotemporal state in a test set. The higher-dimensional and/or spatiotemporal states may be reconstructed using the linear approximation expressed in the equation five “(5)” of FIG. 8. In (5) of FIG. 8, {yᵢ}^{t} may denote measurements of the higher-dimensional and/or spatiotemporal state {x}ᵢ₌₁^{t}; β may denote a learned set of parameters; and {ϵᵢ}^{t} may denote unobserved random variables.

In some embodiments, the reconstruction error of the least squares linear regression model over each higher-dimensional and/or spatiotemporal state can be calculated using (3) of FIG. 8.

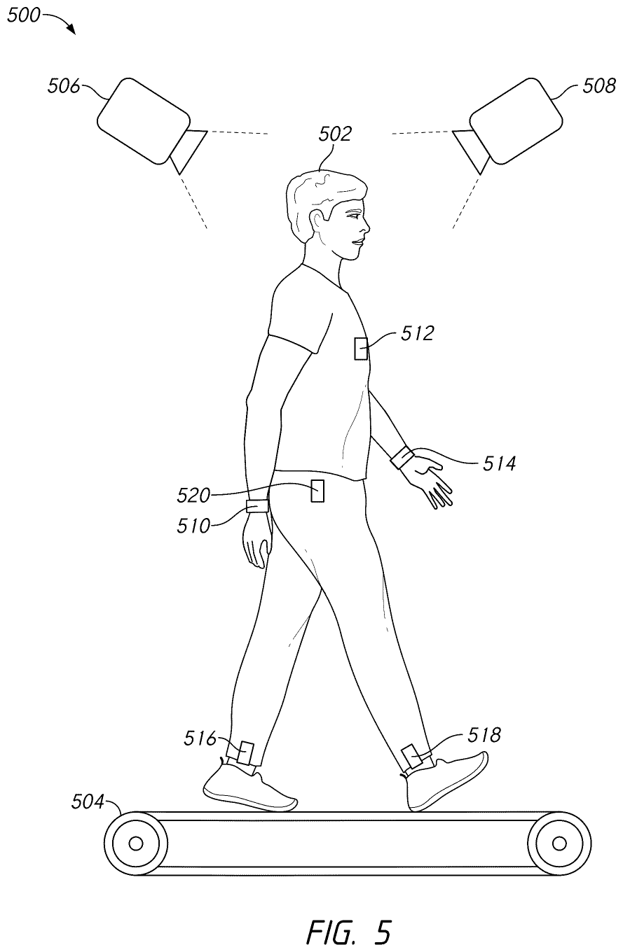

FIG. 5 shows an environment 500 of a user walking on a treadmill, where motions of the user are being measured and/or tracked by one or more sensors, in accordance with examples described herein.

In some embodiments, the environment 500 of FIG. 5 illustrates a laboratory environment that may be used to test, develop, and/or improve the performance of the computing system 102 of FIG. 1. The environment 500 of FIG. 5 may be used to train and/or test the method 200 of FIG. 2, the machine learning model 302 of FIG. 3, and/or the machine learning model 402 of FIG. 4. It is to be understood, however, that the computing system 102, the methods, and/or the various machine learning models described herein can be used in other environments, such as natural environments and/or every-day environments.

In some embodiments, the environment 500 may include a user 502, a treadmill 504, a camera 506, a camera 508, a mobile sensor 510, a mobile sensor 512, a mobile sensor 514, a mobile sensor 516, a mobile sensor 518, a mobile sensor 520, fewer mobile or stationary sensors, additional mobile or stationary (e.g., cameras) sensors, different mobile and/or stationary sensors, and/or sensors located at locations that are not explicitly illustrated in FIG. 5. In some embodiments, one, some, or each of the mobile sensors 510 to 520 may be, or may be included in, an IMU.

In some embodiments, these stationary and/or mobile sensors may measure and/or capture various motions of various portions of the body of the user. By so doing, the computing system 102 of FIG. 1 and/or the method 200 of FIG. 2 can obtain a time series of measurements from at least one mobile sensor and/or a stationary sensor. As is illustrated in FIG. 5, the mobile sensors 510 to 520 can be placed on different portions of the body, as the user walks, jogs, or runs on the treadmill 504.

Although FIG. 5 illustrates the user 502 on a substantially flat treadmill 504, the systems (e.g., the computing system 102 of FIG. 1), methods (e.g., the method 200 of FIG. 2), and/or machine learning models (e.g., machine learning models 110, 302, 402) described herein can be utilized when the user is walking on a tilted treadmill, or when the user is performing another physical activity, such as cycling, swimming, lifting weights, performing aerobic exercises, calisthenics, plyometric exercises, yoga, Pilates, isometric exercises, agility drills, flexibility exercises, etc.

In some embodiments, the time series of measurements using the various sensors, reconstructed additional estimated sensor data, and/or predicted physiological measurements and/or biomarkers can be compared to evaluate the performance of the systems, methods, and/or models.

### Examples

In some embodiments, using any of the machine learning models described herein, the computing system 102 of FIG. 1 may be able to infer from, for example, a sparse set of time series of measurements using mobile and/or stationary sensors to a full set of sensor data. In some embodiments, the computing system 102 of FIG. 1 may obtain a time series of measurements from at least one mobile sensor (e.g., mobile sensors 512 to 520 of FIG. 5) carried by a portion of a body of the user (e.g., the user 502 of FIG. 5, or a plurality of users) during motion of the mobile sensor(s). The computing system 102 of FIG. 1 can then reconstruct additional estimated sensor data using any of the machine learning models described herein based on the time series of measurements. The computing system 102 can then analyze the additional estimated sensor data together with the time series of measurements to predict a physiological measurement(s) and/or a biomarker(s).

The inference and/or the learning of the inference can be performed in a variety of ways, such as training using a single user or group mappings of a plurality of users. For individual training, the computing system 102 of FIG. 1 can utilize the time series of measurements of the biomechanics of the user by utilizing mobile and/or stationary sensors. Then, using any of the machine learning models described herein, the computing system 102 can learn a unique mapping for a personalized reconstruction of additional estimated sensor data. The computing system 102 can then predict (and/or forecast) personalized physiological measurement(s) and/or biomarker(s) by analyzing the additional estimated sensor data together with the time series of measurements.

For group mappings, the computing system 102 of FIG. 1 can utilize the time series of measurements of the biomechanics of a plurality of users, by utilizing mobile and/or stationary sensors associated with each user of the plurality of users. Then, using any of the machine learning models described herein, the computing system 102 can learn a group mapping, and the group mapping can be used to reconstruct additional estimated sensor data for any of the users of the plurality of users or for another user that is not part of the plurality of users. In some aspects, the group mapping can be utilized for a generic user and may not be personalized for any specific user. The computing system 102 can then predict (and/or forecast) physiological measurement(s) and/or biomarker(s) of any user (a member of the plurality of users or a non-member of the plurality of users) by analyzing the additional estimated sensor data together with the time series of measurements. Note that the time series of measurements were measured using at least one mobile sensor associated with any user. The group mapping can be useful in deploying and/or utilizing an already-trained machine learning model to be utilized by any user in a future time period. For example, a user can purchase a device, such as a device including an IMU. The device may include and/or utilize an already-trained machine learning model as described herein. The user can then use the IMU to predict physiological measurement(s) and/or biomarker(s) of the user.

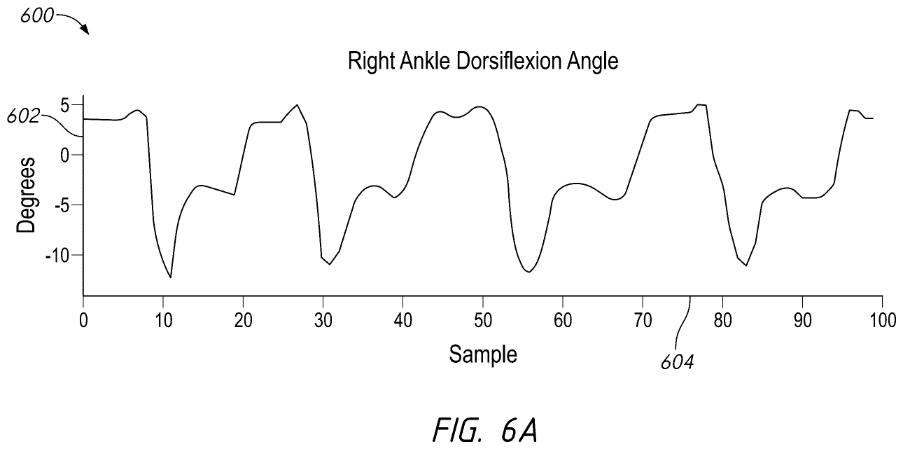

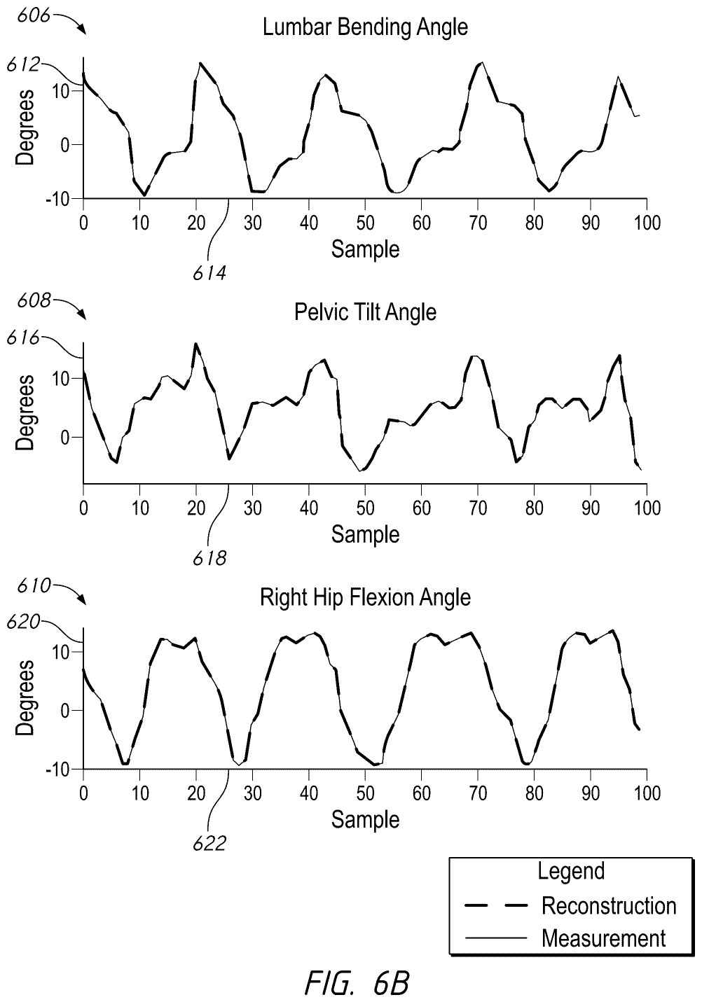

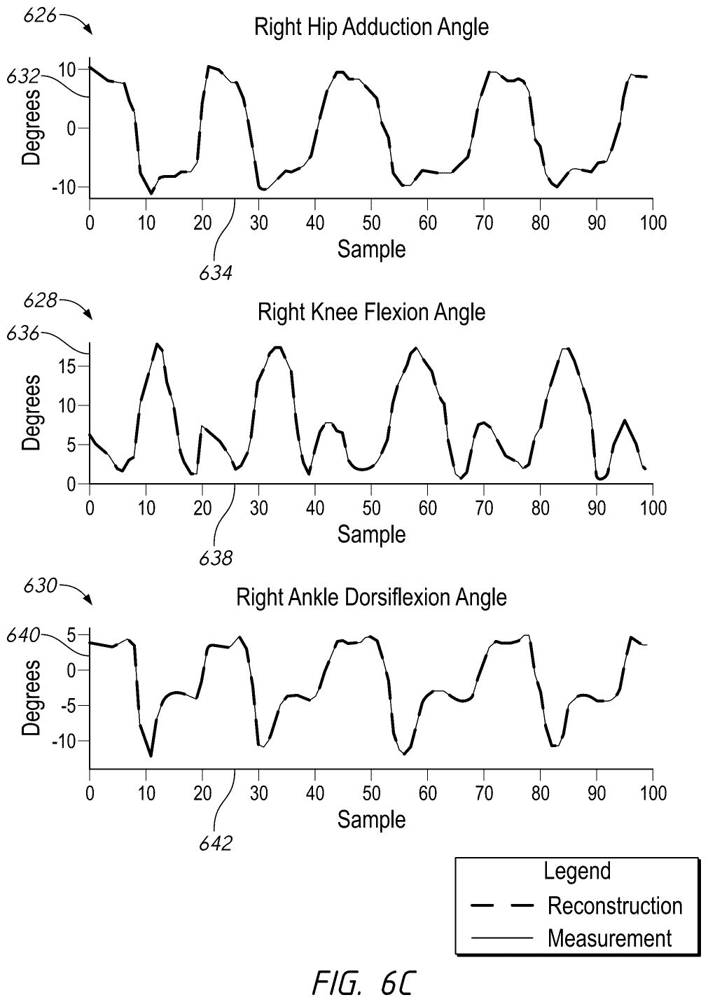

FIG. 6A shows a graph 600 of measurements of a right ankle dorsiflexion angle, where the measurements may be part of a kinematics dataset, in accordance with examples described herein.

In some embodiments, for example, in a laboratory environment, the graph 600 of FIG. 6A shows an angle 602 (in degrees) of measurement samples 604 of the right ankle dorsiflexion angle of a person (e.g., the user 502 of FIG. 5). The measurements (e.g., a time series of measurements) may be captured using camera(s) (e.g., the cameras 506 and/or 508 of FIG. 5), infrared sensor(s), proximity sensor(s), laser(s), LiDAR(s), radar(s), and/or other devices capable of measuring the right ankle dorsiflexion angle of a person (user). For the sake of clarity, graph 600 does not include reconstructed additional estimated sensor data. Therefore, a machine learning model may not be needed to plot the data of the graph 600. Accordingly, in this example, cameras are used to track one or more markers on a user to obtain the graph shown in FIG. 6A.

FIG. 6B shows a graph 606, a graph 608, and a graph 610, and FIG. 6C shows a graph 626, a graph 628, and graph 630 of reconstructed angles and measured angles of various body parts of the user, where the reconstructed angles are outputs of a machine learning model, and where the reconstructed angles and the measured angles may be part of the kinematics dataset, in accordance with examples described herein. In FIG. 6B and FIG. 6C, the measured angles are shown with a solid line, the reconstructed angles are shown with a dashed line, and these measured angles and reconstructed angles are plotted on the same graph for a specific body part of the person.

The measured lines are indicative of test examples where the depicted parameters were directly measured using cameras, markers, and/or other sensors carried by the user. The reconstruction lines are indicative of output of an example machine learning model arranged in accordance with examples described herein (e.g., the machine learning model(s) shown and described with reference to FIGS. 1-5). The machine learning model received the right angle dorsiflexion time series measurements of FIG. 6A, and output the reconstructed data shown as right hip adduction angle, right knee flexion angle, and right ankle dorsiflexion angle. Accordingly, note that in this example, a trained machine learning model received as input a time series measurement of a user's right ankle dorsiflexion angle. Based on this input, the machine learning model output reconstructed data for the lumbar bending angle, pelvic tilt angle, right hip flexion angle, right hip adduction angle, right knee flexion angle, and right ankle dorsiflexion angle. As shown, in this implemented example, there was good agreement between directly measured data and reconstructed data. In normal operation, the reconstructed data may not be directly measured.

The reconstructed data shown in FIGS. 6B and 6C may be analyzed in accordance with techniques described herein to determine one or more biomarkers-such as gait length, loading, fall probability, and/or other biomechanical metrics. While in the example of FIGS. 6A-6C, reconstructed data was reconstructed for a same time period as input measured data (e.g., samples 0-100), in other examples, data may be reconstructed for time periods before and/or after the measured input data. Accordingly, future biomechanical parameters may be predicted using models described herein.

In some embodiments, in human anatomy, kinematics, and/or biomechanics, movements can be categorized into several planes, such as a sagittal plane, a frontal plane, and/or a transverse plane. For the sake of brevity, the reconstructed angle data shown in FIG. 6B and FIG. 6C include only sagittal-plane kinematics data. It is to be understood that the systems and methods described herein may use kinematics datasets of other planes.

In some embodiments, the graph 606 of FIG. 6B shows an angle 612 (in degrees) of measurement samples 614 and reconstructed samples 614 of a lumber bending angle of the person (e.g., the user 502 of FIG. 5). The graph 608 of FIG. 6B shows an angle 616 (in degrees) of measurement samples 618 and reconstructed samples 618 of a pelvic tilt angle of the person. The graph 610 of FIG. 6B shows an angle 620 (in degrees) of measurement samples 622 and reconstructed samples 622 of a right hip flexion angle of the person.

In some embodiments, the graph 626 of FIG. 6C shows an angle 632 (in degrees) of measurement samples 634 and reconstructed samples 634 of a right hip adduction angle of the person (e.g., user 502 of FIG. 5). The graph 628 of FIG. 6C shows an angle 636 (in degrees) of measurement samples 638 and reconstructed samples 638 of a right knee flexion angle of the person. The graph 630 of FIG. 6C shows an angle 640 (in degrees) of measurement samples 642 and reconstructed samples 642 of a right ankle dorsiflexion angle of the person.

In some embodiments, the measured angles (illustrated with solid lines in FIG. 6B and FIG. 6C) shown in the graph 606, graph 608, graph 610, graph 626, graph 628, and graph 630 may be captured using camera(s) (e.g., cameras 506 and/or 508 of FIG. 5), infrared sensor(s), proximity sensor(s), laser(s), LiDAR(s), radar(s), and/or other devices capable of measuring these angles of the various body parts of the person (user). These measurements may be, or include, a time series of measurements of the various body parts of the person.

In some embodiments, the reconstructed angles (illustrated with dashed lines in FIG. 6B and FIG. 6C) shown in the graph 606, graph 608, graph 610, graph 626, graph 628, and graph 630 may be reconstructed using any of the systems and methods described herein. For example, the computing system 102 of FIG. 1 may obtain a time series of measurements of a first body part (e.g., the right ankle dorsiflexion angle of FIG. 6A) as the user performs an activity. The computing system 102 can then reconstruct additional estimated sensor data using any of the machine learning models described herein based on the time series of measurements. These additional estimated sensor data may be associated with the same body part or with additional body parts. The computing system 102 can analyze the additional estimated sensor data together with the time series of measurements to predict a physiological measurement and/or a biomarker of interest to the user. In some embodiments, even though the time series of measurements may be associated with a specific body part of the user, the reconstructed additional estimated sensor data may be associated with that specific body part or other body parts of the user. Therefore, in some embodiments, the computing system 102 may reconstruct and/or predict at least one higher-dimensional body movement compared to the time series of measurements.

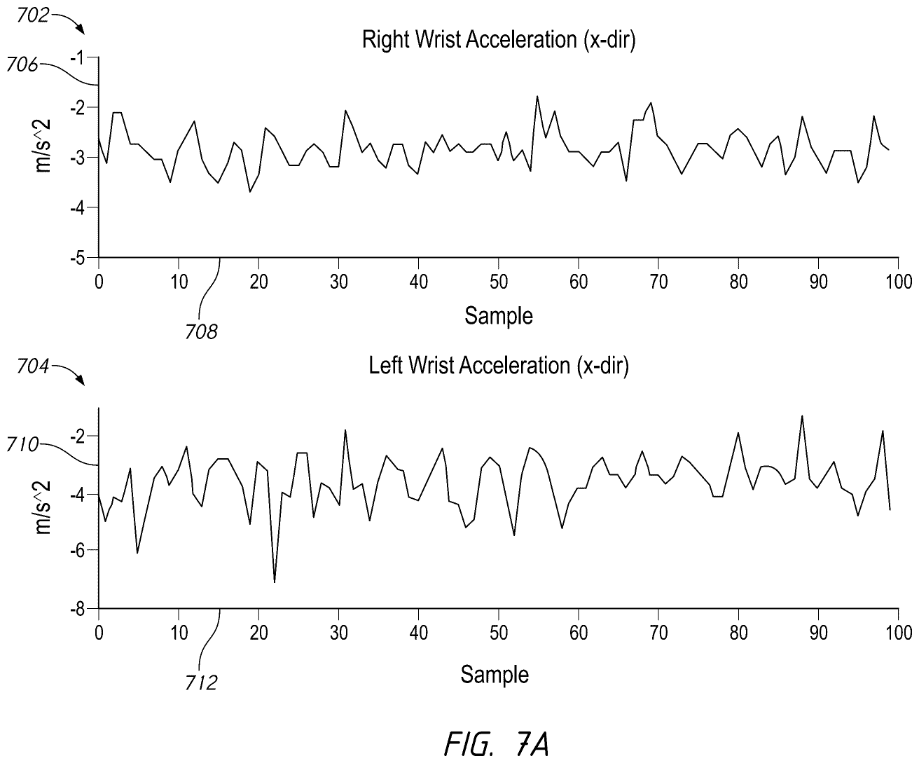

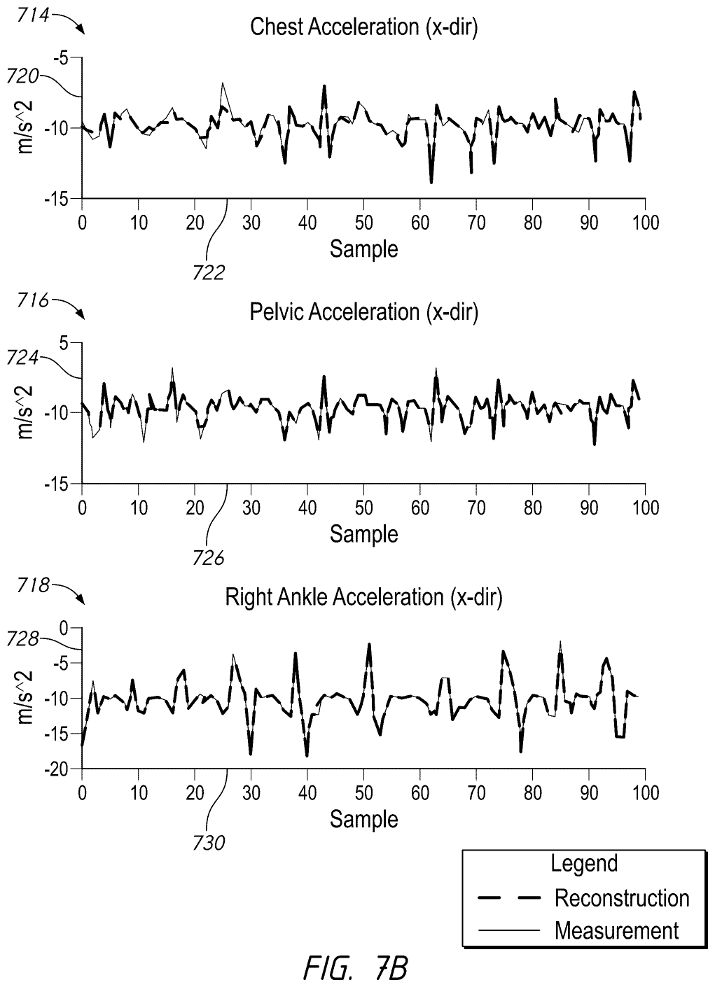

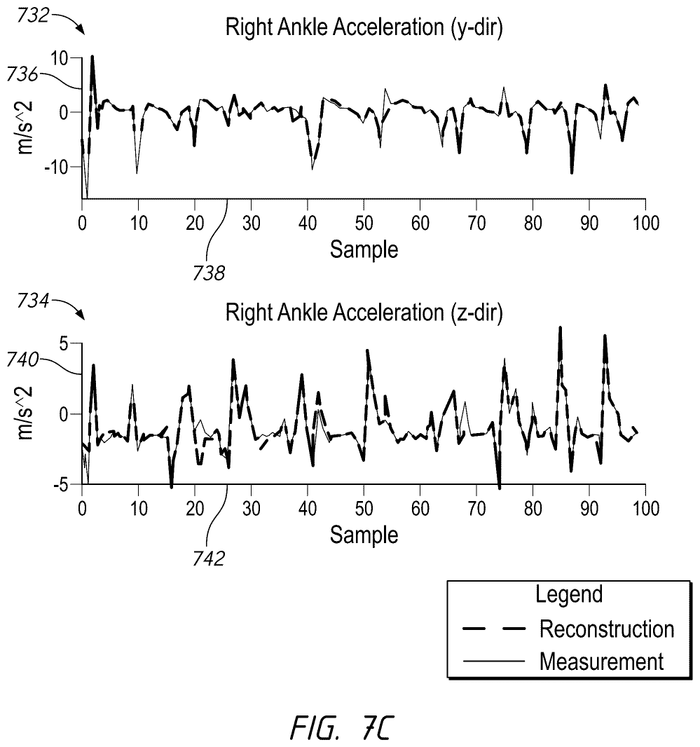

FIG. 7A shows a graph 702 of measurements of a right wrist acceleration of the user, and a graph 704 of measurements of a left wrist acceleration of the user, where the measurements may be part of an IMU dataset, in accordance with examples described herein.

In some embodiments, the graph 702 of FIG. 7A shows an acceleration 706 value(s) (in m/s²) of measurement samples 708 of the right wrist acceleration in the x-direction of a person (e.g., the user 502 of FIG. 5). In some embodiments, the measurements shown in graph 702 (e.g., a time series of measurements) may be captured using the mobile sensor 510 of FIG. 5, where the mobile sensor 510 may be, or may be included in, an IMU. For example, the user may carry or wear this IMU on their right wrist, such as a smartphone.

In some embodiments, the graph 704 of FIG. 7A shows an acceleration 710 value(s) (in m/s²) of measurement samples 712 of the left wrist acceleration in the x-direction of the person (e.g., the user 502 of FIG. 5). In some embodiments, the measurements (e.g., a time series of measurements) may be captured using the mobile sensor 514 of FIG. 5, where the mobile sensor 514 may be, or may be included in, an IMU. For example, the user may carry or wear this IMU on their left wrist, such as a smartphone.

For the sake of clarity, the graph 702 and the graph 704 of FIG. 7A do not include reconstructed additional estimated sensor data. Therefore, a machine learning model may not be needed to plot the data of the graph 702 and/or the graph 704.

FIG. 7B shows a graph 714, a graph 716, and a graph 718, and FIG. 7C shows a graph 732 and a graph 734 of reconstructed accelerations and measured accelerations of various body parts of the user, where the reconstructed accelerations are outputs of the machine learning model, and where the reconstructed and measured accelerations may be part of the IMU dataset, in accordance with examples described herein. In FIG. 7B and FIG. 7C, the measured accelerations are shown with a solid line, the reconstructed accelerations are shown with a dashed line, and these measured accelerations and reconstructed accelerations are plotted on the same graph for a specific body part of the person.

In some embodiments, the graph 714 of FIG. 7B shows an acceleration 720 value(s) (in m/s²) of measurement samples 722 and reconstructed samples 722 of a chest acceleration in the x-direction of the person (e.g., the user 502 of FIG. 5). The graph 716 of FIG. 7B shows an acceleration 724 value(s) (in m/s²) of measurement samples 726 and reconstructed samples 726 of a pelvic acceleration in the x-direction of the person. The graph 718 of FIG. 7B shows an acceleration 728 value(s) (in m/s²) of measurement samples 730 and reconstructed samples 730 of a right ankle acceleration in the x-direction of the person.

In some embodiments, the graph 732 of FIG. 7C shows an acceleration 736 value(s) (in m/s²) of measurement samples 738 and reconstructed samples 738 of a right ankle acceleration in the y-direction of the person (e.g., the user 502 of FIG. 5). The graph 734 of FIG. 7C shows an acceleration 740 value(s) (in m/s²) of measurement samples 742 and reconstructed samples 742 of a right ankle acceleration in the z-direction of the person.

In some embodiments, the measured accelerations (illustrated with solid lines in FIG. 7B and FIG. 7C) shown in the graph 714, the graph 716, the graph 718, the graph 732, and the graph 734 may be measured using various mobile sensors and/or IMUs, such as the mobile sensor 510, the mobile sensor 512, the mobile sensor 514, the mobile sensor 516, the mobile sensor 518, and/or the mobile sensor 520 of FIG. 5. These measurements may be, or include, a time series of measurements of the various body parts of the person.

In some embodiments, the reconstructed accelerations (illustrated with dashed lines in FIG. 7B and FIG. 7C) shown in the graph 714, the graph 716, the graph 718, the graph 732, and the graph 734 may be reconstructed using any of the systems and methods described herein. For example, the computing system 102 of FIG. 1 may obtain a time series of measurements of a first body part (e.g., the right wrist acceleration of FIG. 7A) as the user performs an activity, while utilizing an IMU. Additionally, or alternatively, the computing system 102 of FIG. 1 may obtain a time series of measurements of a second body part (e.g., the left wrist acceleration of FIG. 7A) as the user performs the activity, while using the same IMU or another IMU. The computing system 102 can then reconstruct additional estimated sensor data using any of the machine learning models described herein based on the time series of measurements. These additional estimated sensor data may be associated with the same body part or with additional body parts. The computing system 102 can analyze the additional estimated sensor data together with the time series of measurements to predict a physiological measurement and/or a biomarker of interest to the user. In some embodiments, even though the time series of measurements may be associated with a specific body part of the user, the reconstructed additional estimated sensor data may be associated with that specific body part or other body parts of the user. Therefore, in some embodiments, the computing system 102 may reconstruct and/or predict at least one higher-dimensional body movement compared to the time series of measurements.

The measured lines are indicative of test examples where the depicted parameters were directly measured using IMUs and/or other sensors carried by the user. The reconstruction lines are indicative of output of an example machine learning model arranged in accordance with examples described herein (e.g., the machine learning model(s) shown and described with reference to FIGS. 1-5). The machine learning model received the right and left wrist acceleration time series measurements of FIG. 7A, and output the reconstructed data shown as chest acceleration, pelvic acceleration, right ankle acceleration (x-direction), right ankle acceleration (y-direction), and right ankle acceleration (z-direction). Accordingly, note that in this example, a trained machine learning model received as input a time series measurement of a user's right and left wrist acceleration. Based on this input, the machine learning model output reconstructed data for the chest acceleration, pelvic acceleration, right ankle acceleration (x-direction), right ankle acceleration (y-direction), and right ankle acceleration (z-direction). As shown, in this implemented example, there was good agreement between directly measured data and reconstructed data. In normal operation, the reconstructed data may not be directly measured.

The reconstructed data shown in FIGS. 7B and 7C may be analyzed in accordance with techniques described herein to determine one or more biomarkers-such as gait length, loading, fall probability, and/or other biomechanical metrics. While in the example of FIGS. 7A-7C, reconstructed data was reconstructed for a same time period as input measured data (e.g., samples 0-100), in other examples, data may be reconstructed for time periods before and/or after the measured input data. Accordingly, future biomechanical parameters may be predicted using models described herein.

### Examples

In some embodiments, the SHRED architecture, the SDN architecture, the linear regression, and/or another architecture of the machine learning model can be trained with, or process, a first dataset by using, for example, a time lag of k=120.

In some embodiments, the first dataset may be collected motion data from, for example, 12 non-disabled adults. The 12 non-disabled adults included six males and six females in one implemented example, with ages=23.9±1.8 years; height=1.69±0.10 m; mass=66.5±11.7 kg. These adults may be walking on a treadmill (e.g., the treadmill 504 of FIG. 5) at a speed of, for example, 1.36±0.11 m/s. In some embodiments, these adults may be equipped with passive and/or active markers that may be selectively placed in various portions of their bodies. In such a case, using cameras, such as motion cameras, the camera 506, the camera 508 of FIG. 5, and/or other devices may capture motions of the adults. Consequently, the collected first dataset may include kinematics data and/or electromyography data of one, some, or each of the 12 adults.

In some embodiments, the systems, the machine learning models, and/or methods described herein can learn and/or perform mapping to reconstruct the kinematics and/or electromyography data of the first dataset.

Continuing with the first dataset, in some embodiments, for the individual mapping, the machine learning model can be trained to use a reconstruction mapping for each adult participant, where the machine learning model may be, or include, the SHRED architecture, the SDN architecture, and/or the linear regression model.

Continuing with the first dataset, in some embodiments, for the individual mapping, the system(s), models, and/or machine learning model(s) described herein can be tested to reconstruct various kinematic states, for example, of: (i) three randomly chosen kinematic states (e.g., transverse-plane pelvic rotation angle, medio-lateral pelvis position, right hip adduction angle); (ii) three purposefully chosen kinematic states (e.g., right hip flexion angle, right knee flexion angle, right ankle dorsiflexion angle); (iii) one randomly selected kinematic state (e.g., medio-lateral pelvis rotation); (iv) one purposely selected kinematic state (e.g., right ankle dorsiflexion angle); and/or other kinematic states.

Continuing with the first dataset, in some embodiments, for the group mapping, the machine learning model can be trained to use 11 of the 12 adult participants, to predict a kinematic data (um) for the 12^{th} (e.g., hold-out) adult participant. In some embodiments, for the group mapping, a machine learning model and/or a model may be, or may be trained by using, a dynamic deep learning algorithm and/or architecture, a SHRED architecture, an SDN architecture, a linear regression model, etc.

Continuing with the first dataset, in some embodiments, for the group mapping, the system(s), models, and/or machine learning model(s) described herein can be tested to reconstruct various kinematic states, for example, of: (i) three purposely selected kinematic states (e.g., right hip flexion angle, right knee flexion angle, right ankle dorsiflexion angle); (ii) one purposely selected kinematic state (e.g., right ankle dorsiflexion angle); and/or other kinematic states.

Continuing with the first dataset, Table 1, qualitatively, shows various trained models/architectures mapping results between a sparse set of measurements (e.g., time series of measurements) to reconstruct additional, expanded, full, and/or higher-dimensional body (and/or spatiotemporal state) movement kinematics data.

TABLE 1
First Dataset (e.g., kinematics)

| | | MSE (±s.d.), | | |
|---|---|---|---|---|
| | | Accuracy and/or Precision | | |
| Type | Input Sensors | SHRED | SDN | Linear |
| Individual | Three (3) random | Highest | Second | Third |
| | (e.g., transverse-plane | | Highest | Highest |
| | pelvis rotation angle, | | | |
| | medio-lateral pelvis position, | | | |
| | right hip adduction angle) | | | |
| | Three (3) purposeful | Highest | Second | Third |
| | (e.g., right hip flexion angle, | | Highest | Highest |
| | right knee flexion angle, | | | |
| | right ankle dorsiflexion angle) | | | |
| | One (1) random | Highest | Second | Third |
| | (e.g., medio-lateral pelvis | | Highest | Highest |
| | position) | | | |
| | One (1) purposeful | Highest | Second | Third |
| | (e.g., right ankle dorsiflexion | | Highest | Highest |
| | angle) | | | |
| Group | Three (3) purposeful | Highest | Second | Third |
| | (e.g., right hip flexion angle, | | Highest | Highest |
| | right knee flexion angle, | | | |
| | right ankle dorsiflexion angle) | | | |
| | One (1) purposeful | Highest | Second | Third |
| | (e.g., right ankle dorsiflexion | | Highest | Highest |
| | angle) | | | |

Although Table 1 displays qualitative results, generally, for a higher accuracy, an MSE value may be lower. Similarly, for a higher precision, a standard deviation (s.d.) may be lower.

In some embodiments, the SHRED architecture, the SDN architecture, the linear regression, and/or another architecture of the machine learning model can be trained with, or process, a second dataset by using a time lag of k=128. Other examples may include training these architectures using other dataset(s) and/or other k values.

In some embodiments, the second dataset may be collected motion data from, for example, ten (10) non-disabled adults. The ten non-disabled adults may include eight males and two females, with ages=27.4±4.5 years; height=1.76±0.09 m; mass=69.1±9.9 kg. These adults may be wearing and/or utilizing IMUs, and these adults may be performing a variety of steady-state and/or time-varying activities. For example, the adults may be utilizing, carrying, and/or wearing IMUs (e.g., accelerometers) on their left ankle, right ankle, left hip (or right hip), center of their chest, etc. As another example, the adults may utilize, carry, and/or wear bilateral wristbands, or some other IMUs. These IMUs may measure acceleration in the x-, y-, and z-axes (directions).

In some embodiments, the second dataset may include two different IMU subsets of data. The first subset of the second dataset may include non-wrist IMUs, such as IMUs placed on or near a left ankle, a right ankle, a left hip (or right hip), center of the chest, etc. The second subset of the second dataset may include the bilateral wrist IMUs, in addition to the other IMUs.

In some embodiments, the systems, the machine learning models, and/or methods described herein can learn and/or perform mapping to reconstruct the first and the second subsets of the second dataset, where these subsets include IMU data (e.g., time series of measurements).

Continuing with the second dataset, in some embodiments, for the individual mapping, the machine learning model can be trained to use a reconstruction mapping for each of adult participants, where the machine learning model may be, or include, a dynamic deep learning algorithm, the SHRED architecture, the SDN architecture, and/or the linear regression model.

Continuing with the first subset of the second dataset, in some embodiments, for the individual mapping, the system(s), models, and/or machine learning model(s) described herein can be tested to reconstruct various IMU signal and/or data, such as: (i) triaxial left hip accelerations; (ii) triaxial left ankle accelerations; (iii) triaxial center chest accelerations; (iv) single-axis (e.g., x-direction) left hip accelerations; (v) single-axis (e.g., x-direction) left ankle accelerations; (vi) single-axis (e.g., x-direction) center chest accelerations; and/or other IMU data.

Continuing with the second dataset (e.g., for the first and/or second subsets of the second dataset and/or IMU-based dataset), in some embodiments, for the group mapping, the machine learning model can be trained to use nine (9) of the ten (10) adult participants, to predict an IMU data (um) for the 10^{th} (e.g., hold-out) adult participant, when the hold-out participant utilizes at least one mobile sensor. In some embodiments, the machine learning model and/or a model may be, or may be trained by using, a dynamic deep learning algorithm and/or architecture, a SHRED architecture, an SDN architecture, a linear regression model, etc.

Continuing with the first subset of the second dataset, in some embodiments, for the group mapping, the system(s), models, and/or machine learning model(s) described herein can be tested to reconstruct various IMU signals/data, for example, of: (i) triaxial left hip accelerations; (ii) triaxial left ankle accelerations; (iii) triaxial center chest accelerations; (iv) single-axis left hip accelerations (e.g., in the x-direction); (v) single-axis left ankle acceleration (e.g., in the x-direction); (vi) single-axis center chest acceleration (e.g., in the x-direction); and/or other IMU signals/data.

Continuing with the second subset (e.g., wrist-related) of the second dataset, in some embodiments, for the individual mapping, the machine learning model can be trained to use a reconstruction mapping for each adult participant, where the machine learning model may be, or include, a dynamic deep learning algorithm, the SHRED architecture, the SDN architecture, and/or the linear regression model. In some embodiments, left and right wrist IMU signals (e.g., time series of measurements) may be used as inputs to each model described herein to reconstruct other or additional (e.g., non-wrist) IMU data.

Continuing with the second dataset, Table 2, qualitatively, shows various trained models/architectures mapping results between a sparse set of measurements (e.g., time series of measurements) to reconstruct additional, expanded, full, and/or higher-dimensional body (and/or spatiotemporal state) movement IMU data.

TABLE 2
Second Dataset (e.g., IMU signals/data)

| | | | MSE (±s.d.) | | |
|---|---|---|---|---|---|
| | | | Accuracy and/or Precision | | |
| Type | | Input Sensors | SHRED | SDN | Linear |
| Non-wrist | Individual | Triaxial Pelvic | Highest | Second | Third |
| | | Acceleration | | Highest | Highest |
| | | Triaxial Ankle | Highest | Second | Third |
| | | Acceleration | | Highest | Highest |
| | | Triaxial Chest | Highest | Second | Third |
| | | Acceleration | | Highest | Highest |
| | | Single-axis Pelvic | Highest | Second | Third |
| | | Acceleration (x-dir) | | Highest | Highest |
| | | Single-axis Ankle | Highest | Second | Third |
| | | Acceleration (x-dir) | | Highest | Highest |
| | | Single-axis Chest | Highest | Second | Third |
| | | Acceleration (x-dir) | | Highest | Highest |
| | Group | Triaxial Pelvic | Highest | Second | Third |
| | | Acceleration | | Highest | Highest |
| | | Triaxial Ankle | Highest | Second | Third |
| | | Acceleration | | Highest | Highest |
| | | Triaxial Chest | Highest | Second | Third |
| | | Acceleration | | Highest | Highest |
| | | Single-axis Pelvic | Highest | Second | Third |
| | | Acceleration (x-dir) | | Highest | Highest |
| | | Single-axis Ankle | Highest | Second | Third |
| | | Acceleration (x-dir) | | Highest | Highest |
| | | Single-axis Chest | Highest | Second | Third |
| | | Acceleration (x-dir) | | Highest | Highest |
| Wrist | Individual | Bilateral Triaxial | Highest | Second | Third |
| | | Acceleration | | Highest | Highest |
| | Group | Bilateral Triaxial | Highest | Second | Third |
| | | Acceleration | | Highest | Highest |

Although Table 1 displays qualitative results, generally, for a higher accuracy, an MSE value may be lower. Similarly, for a higher precision, a standard deviation (s.d.) may be lower.

FIG. 8 shows a list of mathematical equations or expressions, in accordance with examples described herein. In FIG. 8, (1) denotes equation one; (2) denotes mathematical expression two; (3) denotes equation three; (4) denotes mathematical expression four; and (5) denotes equation five, as described and/or utilized herein.

From the foregoing it will be appreciated that, although specific embodiments have been described herein for purposes of illustration, various modifications may be made while remaining within the scope of the claimed technology. Examples described herein may refer to various components as “coupled” or signals as being “provided to” or “received from” certain components or nodes. It is to be understood that in some examples the components are directly coupled one to another, while in other examples, the components are coupled with intervening components disposed between them.

Similarly, signals or communications may be provided directly to and/or received directly from the recited components without intervening components, but also may be provided to and/or received from the certain components through intervening components.

What is claimed is:

1. A method comprises:
obtaining a time series of measurements from at least one mobile sensor carried by a portion of a body during motion of the at least one mobile sensor;
reconstructing additional estimated sensor data using a machine learning model based on the time series of measurements; and
analyzing the additional estimated sensor data together with the time series of measurements to predict a physiological measurement, a biomarker, or combinations thereof.

2. The method of claim 1, wherein said analyzing further comprises selecting a subset of the additional estimated sensor data together with the time series of measurements, wherein the selected subset of the additional estimated sensor data is related to the physiological measurement, the biomarker, or combinations thereof.

3. The method of claim 1 further comprises determining at least one higher-dimensional body movement compared to the time series of measurements from the at least one mobile sensor carried by the portion of the body.

4. The method of claim 1, wherein the machine learning model comprises a sequential model for encoding time sequences followed by a decoder network mapping an output of the sequential model to a final output.

5. The method of claim 1 further comprises training the machine learning model using a plurality of users during a first time period.

6. The method of claim 5, wherein said obtaining, said reconstructing, and said analyzing are associated with a single user during a second time period.

7. The method of claim 6, wherein an identity of the single user differs from identities of each user of the plurality of users.

8. The method of claim 1 further comprises training the machine learning model using a user during a first time period, a first environmental setting, or combinations thereof.

9. The method of claim 8, wherein said obtaining, said reconstructing, and said analyzing are associated with the user during a second time period, a second environmental setting, or combinations thereof.

10. The method of claim 1, wherein the at least one mobile sensor comprises inertial measurement units.

11. The method of claim 1, wherein the at least one mobile sensor is embedded in a wearable device.

12. The method of claim 1, wherein the physiological measurement, the biomarker, or combinations thereof comprise a stride length.

13. A computing system comprises:
a processor; and
a non-transitory computer-readable storage medium storing instructions that, when executed by the processor, cause the computing system to perform operations comprising:
obtain a time series of measurements from an at least one mobile sensor carried by a portion of a body during motion of the at least one mobile sensor;
reconstruct additional estimated sensor data using a machine learning model based on the time series of measurements; and
analyze the additional estimated sensor data together with the time series of measurements to predict a physiological measurement, a biomarker, or combinations thereof.

14. The computing system of claim 13, wherein the machine learning model comprises a time sequence model and a decoder, and wherein the time sequence model increases an accuracy of the reconstructed additional estimated sensor data.

15. The computing system of claim 13, wherein a user comprises the portion of the body, and wherein the instructions, when executed by the processor, cause the computing system to perform operations comprising:
map biomechanical measurements during motion of the portion of the body; and
train the machine learning model to reconstruct the additional estimated sensor data for the user based on the time series of measurements from the at least one mobile sensor.

16. The computing system of claim 13, wherein the instructions, when executed by the processor, cause the computing system to perform operations comprising:
map biomechanical measurements of a plurality of users;
train the machine learning model to reconstruct the one or more additional estimated sensor data; and
analyze the additional estimated sensor data together with the time series of measurements to predict the physiological measurement, the biomarker, or combinations thereof of another user, wherein an identity of the other user differs from an identity of each user of the plurality of users, and wherein the other user comprises the portion of the body.

17. The computing system of claim 13 further comprises a display screen to display the physiological measurement, the biomarker, or combinations thereof.

18. The computing system of claim 13 comprises a mobile electronic device, and wherein the mobile electronic device comprises the at least one mobile sensor.

19. The computing system of claim 13 further comprises an interface, wherein the interface communicatively couples the computing system to the at least one mobile sensor.

20. The computer system of claim 13, wherein the at least one mobile sensor comprises a thermocouple, a thermistor, a resistance temperature detector, a pressure sensor, a light sensor, a motion sensor, a proximity sensor, a gas sensor, an air quality sensor, a pH sensor, a humidity sensor, a magnetic sensor, a biometric sensor, inertial measurement units, or combinations thereof.
